(19) United States
(12) Patent Application Publication
(10) Pub. No.: US 2013/0217778 A1
(43) Pub. Date: Aug. 22, 2013

(54) METHODS AND COMPOSITIONS FOR THE IMPROVEMENT OF SKELETAL MUSCLE FUNCTION IN A MAMMAL

(71) Applicants: Herman H. Vandenburgh, Steamboat Springs, CO (US); Janet E. Shansky, Warren, RI (US); Frank Benesch-Lee, Cambridge, MA (US)

(72) Inventors: Herman H. Vandenburgh, Steamboat Springs, CO (US); Janet E. Shansky, Warren, RI (US); Frank Benesch-Lee, Cambridge, MA (US)

(21) Appl. No.: 13/811,103

(22) PCT Filed: Jun. 29, 2011
(86) PCT No.: PCT/US11/42295

§ 371 Date: May 7, 2013

Related U.S. Application Data
(60) Provisional application No. 61/366,330, filed on Jul. 21, 2010.

(51) Int. Cl. A61K 31/155

(52) U.S. Cl. CPC A61K 31/155
USPC 514/635; 564/233

(57) ABSTRACT

The present invention is directed to the treatment of muscular dysfunction or increasing muscle strength and/or decreasing muscle fatigue in a subject using a composition that includes a biguanide or a pharmaceutically acceptable salt thereof, e.g., at a low dosage.

### STATEMENT AS TO FEDERALLY SPONSORED RESEARCH

This invention was made with government support under grant number NIH R44 NS059098. The government has certain rights in the invention.

### BACKGROUND OF THE INVENTION

In general, the invention relates to methods of using a biguanide to treat muscular dysfunction, increase muscle strength, and/or reduce muscle fatigue. The invention also features pharmaceutical compositions formulated with low dosages of a biguanide.

Progressive skeletal muscle weakness and fatigue accompany numerous human diseases and disorders including, e.g., cancer, acquired immune deficiency syndrome (AIDS), advanced organ failure (e.g., heart, liver, and kidney failure), chronic obstructive pulmonary disease (COPD), immobilization or disuse atrophy, burns, incontinence, sepsis, aging, and neuromuscular diseases (e.g., muscular dystrophies). Certain therapies may slow or reverse a decline in muscle mass or function including, e.g., hormonal interventions, exercise and physical therapy, nutritional supplements, corticosteroids, and progestational agents. However, there exists no established clinical regimen for treating muscular dysfunction.

There exists a need in the art for improved methods and compositions for treating muscular dysfunction, increasing muscle strength, and/or reducing muscle fatigue.

### SUMMARY OF THE INVENTION

The present invention is directed to the treatment of muscular dysfunction or increasing muscle strength and/or decreasing muscle fatigue in a subject using a composition that includes a biguanide or a pharmaceutically acceptable salt thereof at a low dosage.

In a first aspect, the invention features a method of treating muscular dysfunction in a subject in need thereof by administering to a subject a therapeutically effective amount of a biguanide or a pharmaceutically acceptable salt thereof. Muscular dysfunction may be associated with, for example, muscular dystrophy (e.g., Duchenne muscular dystrophy), sarcopenia, cachexia, cancer, acquired immune deficiency syndrome, advanced organ failure, chronic obstructive pulmonary disease, rhabdomyolysis, disuse atrophy, incontinence, sepsis, neuromuscular disease, and congenital myopathy. Biguanides and pharmaceutically acceptable salts thereof may also be used counter muscle dysfunction and muscle fatigue in subjects being treated with one or more statins (e.g., atorvastatin, rosuvastatin, lovastatin simvastatin, pravastatin, cerivastatin, or fluvastatin).

In another aspect, the invention features a method of increasing muscle strength or decreasing muscle fatigue in a subject by administering to a subject a therapeutically effective amount of a biguanide or a pharmaceutically acceptable salt thereof. The muscle being treated may be skeletal muscle.

In certain embodiments, the therapeutically effective amount of a biguanide administered to a subject results in a concentration between about 0.0000001 μg/ml to about 10.0 μg/ml, about 0.0000001 μg/ml to about 1.0 μg/ml, about 0.0000001 μg/ml to about 0.1 μg/ml, about 0.0000001 μg/ml to about 0.01 μg/ml, about 0.0000001 μg/ml to about 0.001 μg/ml, about 0.0000001 μg/ml to about 0.0001 μg/ml, about 0.0000001 μg/ml to about 0.00001 μg/ml, or about 0.0000001 μg/ml to about 0.000001 μg/ml in blood, serum, or plasma of the subject.

In another aspect, the invention features a method of treating or reducing the likelihood of developing cancer in a subject in need thereof by administering to a subject a low dosage of a biguanide or derivative thereof. And in yet another aspect, the invention features a method of extending the lifespan of a subject by administering to a subject a low dosage of a biguanide or derivative thereof.

In a final aspect, the invention features a pharmaceutical composition that includes a biguanide or pharmaceutically acceptable salt thereof at a dosage unit of less than 250 milligrams.

Biguanides of the invention may be metformin, phenformin, buformin, or proguanil. A biguanide may also be any other biguanide described by formula (I) below. In certain embodiments, biguanides may be administered orally or transdermally to a subject (e.g., a human subject). And, in some embodiments, the subject does not have diabetes or is not taking a corticosteroid.

By “an amount sufficient” is meant the amount of a therapeutic agent (e.g., a biguanide), alone or in combination with another therapeutic agent or therapeutic regimen, required to treat or ameliorate a condition or disorder, e.g., a condition or disorder associated with muscular dysfunction, or symptoms of a condition or disorder in a clinically relevant manner. A sufficient amount of a therapeutic agent (e.g., a biguanide) used to practice the present invention for therapeutic treatment varies depending upon the manner of administration, age, and general health of the subject being treated.

By “biguanide,” “diguanide,” “imidodicarbonimidic diamide,” or “2-carbamimidoylguanidine” is meant a molecule belonging to a class of compounds based upon the biguanide molecule. Exemplary biguanides are described according to Formula (I),

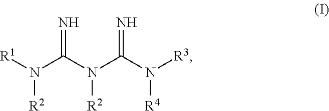

or any stereoisomer or tautomer thereof, or any pharmaceutically acceptable salt, solvate, or prodrug thereof, wherein

each of R¹, R², and R³ is H, optionally substituted C₁₋₁₂ alkyl, optionally substituted C₂₋₁₂ alkenyl, optionally substituted alkoxy, optionally substituted cycloalkyl, optionally substituted alkcycloalkyl, optionally substituted alkcycloalkenyl, optionally substituted alkaryl, optionally substituted alkheteroaryl, optionally substituted aryl, or optionally substituted heteroaryl;

R⁴ is H, optionally substituted C₁₋₁₂ alkyl, optionally substituted C₂₋₁₂ alkenyl, optionally substituted alkoxy, optionally substituted cycloalkyl, optionally substituted alkcycloalkyl, optionally substituted alkcycloalkenyl, optionally substituted alkaryl, optionally substituted alkheteroaryl, optionally substituted aryl, optionally substituted heteroaryl, or has a structure according to substructure A,

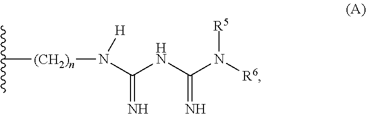

where n is an integer between 2-12, and where optionally one or more carbons in the (CH₂), moiety may be replaced with oxygen, R⁵ is H, optionally substituted C₁₋₆ alkyl, or optionally substituted alkaryl, and R⁶ is optionally substituted aryl or optionally substituted alkaryl,

or R⁴ is —P(═O)(OR^{A})(OR^{B}), where R^{A} and R^{B} are, independently, H, C₁₋₇ alkyl, or a cation (e.g., Na⁺), or R^{A} and R^{B} together form a heterocyclyl;

or R¹ and R², and/or R³ and R⁴, combine to form a heterocyclyl (e.g., aziridine, pyrrolyl, imidazolyl, pyrazolyl, indolyl, indolinyl, pyrrolidinyl, piperazinyl, or piperidyl).

In some embodiments, both R¹ and R² are H. In further embodiments, both R³ and R⁴ are C₁₋₆ alkyl. In other embodiments, R³ is H and R⁴ is optionally substituted alkaryl or alkheteroaryl (e.g., optionally substituted benzyl, phenethyl, or furfuryl). In some embodiments, R³ is H and R⁴ is C₁₋₆ alkyl (e.g., a C₄-C₅ alkyl). In still other embodiments, R³ is C₁₋₆ alkyl (e.g., methyl) and R⁴ is alkaryl (e.g., unsubstituted benzyl or substituted benzyl). In some embodiments, the optionally substituted alkaryl or alkheteroaryl group is benzyl, m-bromobenzyl, p-methoxybenzyl, p-chlorobenzyl, o-chlorobenzyl, p-fluorobenzyl, phenethyl, (p-ClC₆H₄)CH₂CH₂—, (pyridyl)CH₂—, (pyridyl)CH₂CH₂—, furfuryl, (2-furyl)CH₂CH₂—, or (2-thienyl)CH₂—. In other embodiments, the C₁₋₆ alkyl is n-butyl, n-pentyl, 2-methylbutyl, or (CH₃)₂CHCH(CH₃)—.

In other embodiments, both R¹ and R³ are H. In other embodiments, R² is optionally substituted aryl. In still other embodiments, R⁴ is C₁₋₆ alkyl.

In some embodiments, R³ is

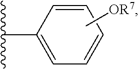

where R⁷ is selected from optionally substituted alkyl, optionally substituted alkenyl, optionally substituted cycloalkyl, or optionally substituted alkaryl. In further embodiments, R¹, R², and R⁴ are each H.

In other embodiments, R³ is

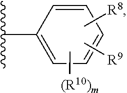

where m is 1, 2, or 3; R⁸ is optionally substituted C₁₋₇ alkyl (e.g., methyl, ethyl, propyl, isopropyl, butyl, isobutyl, sec-butyl, or tert-butyl), or optionally substituted C₂₋₆ alkenyl; R⁹ is halogen (e.g., F, Cl, Br, or I); and each R¹⁰ is, independently, H, halogen, or optionally substituted C₁₋₇ alkyl. In further embodiments, R¹, R², and R⁴ are each H.

In still other embodiments, R³ is

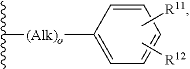

where o is 0 or 1; R¹¹ is an electron-withdrawing group selected from nitro, halogen (e.g., F, Cl, Br, or I), cyano, sulfamoyl, methylsulfonyl (—SO₂CH₃), acetyl (—COCH₃), —CO₂Me, —CCl₃, or a fluoroalkyl (see, e.g., the groups listed in U.S. Pat. No. 3,821,406 at col. 2, lines 43-57); Alk represents an optionally substituted C₁₋₄ alkylene group; and R¹² is H, halogen, nitro, or trifluoromethyl. In further embodiments, R⁴ is H, C₁₋₅ alkyl, C₂₋₅ alkenyl, or optionally substituted alkaryl. In still other embodiments, R¹ and R² are both H.

In still other embodiments, R³ is an alkaryl group having 0, 1, 2, 3, 4, or 5 substituents on the aryl ring that are selected from the group consisting of C₁₋₁₂ alkyl, nitro, amino, halogen, alkoxy, haloalkoxy, haloalkyl, hydroxy, cyano, thiocyanato, carboxylic group, or —SO₂N(R¹³)₂, where each R¹³ is independently a C₁₋₇ alkyl, phenoxy, acyloxy, halophenoxy, phenyl, and halophenyl. In still other embodiments, R¹, R², and R⁴ are each H.

In still other embodiments, R³ is CHCl₂CO₂H and R₄ is H or R⁴ is —P(═O)(OR^{A})(OR^{B}).

In still other embodiments, R³ is an optionally substituted alkyl group having the structure —CH(R¹⁴)CH₂OR¹⁵, where R¹⁴ is optionally substituted C₁₋₄ alkyl, and R¹⁵ is phenyl optionally substituted by 1, 2, 3, 4, or 5 optionally substituted C₁₋₄ alkyl groups.

In still other embodiments, R¹ is a 3,4-dichlorobenzyl group, 4-chlorophenyl group, 3,4-dichlorophenyl group, benzyl group, or 4-chlorobenzyl group, and R³ is an octyl group, 3,4-dichlorobenzyl group, dodecyl group, decyl group, 3-trifluoromethylphenyl group, 4-bromophenyl group, 4-iodophenyl group, 2,4-dichlorophenyl group, 3,4-dichlorophenyl group, 2,3,4-trichlorophenyl group, 3,4-dimethylphenyl group, 3,4-methylenedioxyphenyl group, 4-t-butylphenyl group, 4-ethylthiophenyl group, 1,1,3,3-tetramethylbutyl group, hexyl group, 2-ethoxyethyl group, 2-(2-hydroxyethoxy)ethyl group, 3-diethylaminopropyl group, 3-(2-ethylhexyloxy)-propyl group, (3-isopropoxy)propyl group, (2-diethylamino)-ethyl group, (3-butyl)-propyl group, 3(di-n-butylamino)propyl group, cyclohexylmethyl group, 3-trifluoromethylphenyl group, 4-ethylthiophenyl group, 4-chlorobenzyl group, 2,4-dichlorobenzyl group, 4-acetylaminophenyl group, 3,4-methylenedioxyphenyl group, 3,4-methylenedioxybenzyl group, octyl group, 4-chlorobenzyl group, decyl group, dodecyl group, isobutyl group, 3,4-dichlorophenyl group, or hexyl group. In still other embodiments, R² and R⁴ are both H.

In still other embodiments, R³ is an optionally substituted furfuryl group, where the furfuryl group can have 0, 1, 2, or 3 substituents selected from optionally substituted C₁₋₆ alkyl or optionally substituted C₁₋₆thioalkyl. In further embodiments, R¹, R², and R⁴ are each H.

In some embodiments, the compound of Formula (I) is the hydrochloride, phosphate, sulfate, hydrobromide, salicylate, maleate, benzoate, succinate, ethanedisulfonate, fumarate, glycolate, or clofibrate (2-p-chlorophenoxy-2-methylproprionate) salt.

Biguanides include metformin, phenformin, buformin, and proguanil. Additional biguanides included in the methods and compositions of the present invention include biguanides described in U.S. Pat. Nos. 7,256,218 and 7,396,858, hereby incorporated by reference.

Biguanides useful in the invention include those described herein in any of their pharmaceutically acceptable forms, including isomers such as diastereomers and enantiomers, salts, solvates, and polymorphs thereof, as well as racemic mixtures. Biguanides useful in the invention may also be isotopically labeled compounds. Useful isotopes include hydrogen, carbon, nitrogen, oxygen, phosphorous, fluorine, and chlorine, (e.g., ²H, ³H, ¹³C, ¹⁴C, ¹⁵N, ¹⁸O, ¹⁷O, ³¹P, ³²P, ³⁵S, ¹⁸F, and ³⁶Cl). Isotopically-labeled biguanides can be prepared by synthesizing a compound using a readily available isotopically-labeled reagent in place of a non-isotopically-labeled reagent.

By “condition or disorder associated with muscular dysfunction” is meant any condition or disorder that results, for example, in a decrease in muscle strength or an increase in the rate of muscle fatigue in a subject. Exemplary conditions and disorders associated with muscular dysfunction include, without limitation, muscular dystrophy (e.g., Duchenne, Becker, limb girdle, congenital, facioscapulohumeral, myotonic, oculopharyngeal, distal, spinal, or Emery-Dreifuss muscular dystrophy), Brown-Vialetto-Van Laere syndrome, Fazio-Londe syndrome, Lambert-Eaton myasthenic syndrome, cancer, acquired immune deficiency syndrome (AIDS), advanced organ failure (e.g., heart, liver, or kidney failure), chronic obstructive pulmonary disease (COPD), rhabdomyolysis, tissue hypoxia (e.g., peripheral claudication and exercise intolerance in diabetic subjects), angina, myocardial infarction, disuse atrophy due to prolonged immobility (e.g., resulting from solid organ transplant, joint replacement, stroke, spinal cord injury, recovery from severe burn, or sedentary chronic hemodialysis), Dejerine Sottas syndrome, incontinence, sepsis, aging, neuromuscular diseases (e.g., stroke, Parkinson's disease, multiple sclerosis, myasthenia gravis, Huntington's disease (e.g., Huntington's chorea), or Creutzfeldt-Jakob disease), amyotrophic lateral sclerosis (ALS), post-polio muscular atrophy, chronic muscle fatigue syndrome, or congenital myopathies. Symptoms of muscular dysfunction include, e.g., progressive muscular wasting, low muscle mass, poor balance, frequent falls, walking difficulty, low gait speed, waddling gait, calf deformation, limited range of movement, respiratory difficulty, drooping eyelids, scoliosis, or the inability to walk or lift objects.

By “decrease” or “reduction” is meant to reduce by at least 10%, 20%, 30%, 40%, 50%, 60%, 70%, 75%, 80%, 85%, 90%, 95%, or more. A decrease can refer, for example, to the symptoms of a disorder being treated (e.g., a decrease or reduction in muscle fatigue).

By “improving muscle function” is meant an increase in muscle strength or a decrease in muscle fatigue or rate of muscle fatigue.

By “increase” is meant to augment by at least 10%, 20%, 30%, 40%, 50%, 60%, 70%, 75%, 80%, 85%, 90%, 95%, or more. An increase can refer, for example, to the symptoms of the disorder being treated (e.g., an increase in muscle strength).

By “increasing muscle strength” is meant an increase in the ability of muscle tissue to generate greater maximal tetanic force, resulting in increased ability to, for example, lift objects, move about, or participate in or maintain participation in physical activity (e.g., walking or other exercise).

By “low dosage” is meant at least 5% less (e.g., at least 10%, 20%, 50%, 80%, 90%, or 95%) than the lowest standard recommended dosage of a particular compound formulated for a given route of administration for the treatment of any disease or condition.

By “muscular dysfunction” is meant a decrease in the physiological function of a muscle (e.g., strength, endurance, or agility). By “muscle function” is meant the ability of muscle to perform a physiologic function, such as contraction, as measured by the amount of force generated during either twitch or tetanus. Methods for assessing muscle function are well known in the art and include, but are not limited to, measurements of muscle mass, grip strength, motion or strength tests, tissue histology (e.g., E&A staining, or collagen III staining), or tissue imaging.

By “muscle wasting” or “muscle atrophy” is meant a decrease in muscle mass in a subject and the resulting decrease in muscle strength and/or increase in muscle fatigue.

By “pharmaceutical composition” is meant a composition that includes a therapeutic agent (e.g., a biguanide or a pharmaceutically acceptable salt thereof) formulated with a pharmaceutically acceptable excipient and manufactured for the treatment or prevention of a disorder in a subject. Pharmaceutical compositions can be formulated, for example, for oral administration in unit dosage form (e.g., a tablet, capsule, caplet, gel-cap, or syrup), for topical administration (e.g., as a cream, gel, lotion, patch, or ointment), for intravenous administration (e.g., as a sterile solution, free of particulate emboli, and in a solvent system suitable for intravenous use), or for any other formulation described herein.

By “pharmaceutically acceptable carrier” is meant a carrier that is physiologically acceptable to the treated subject while retaining the therapeutic properties of the therapeutic agent (e.g., a biguanide or derivative thereof) with which it is administered. One exemplary pharmaceutically acceptable carrier substance is physiological saline. Other physiologically acceptable carriers and their formulations are known to one skilled in the art.

By “pharmaceutically acceptable salt” is meant salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the compounds of the invention, or separately by reacting the free base function with a suitable organic acid. Representative acid addition salts include, e.g., acetate, ascorbate, aspartate, benzoate, citrate, digluconate, fumarate, glucoheptonate, glycerophosphate, hemisulfate, heptonate, hexanoate, hydrobromide, hydrochloride, hydroiodide, lactate, malate, maleate, malonate, mesylate, oxalate, phosphate, succinate, sulfate, tartrate, thiocyanate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

The term “alkaryl,” as used herein, represents an aryl group, as defined herein, attached to the parent molecular group through an alkylene group, as defined herein. Exemplary unsubstituted alkaryl groups are of from 7 to 16 carbons. In some embodiments, the alkylene and the aryl each can be further substituted with 1, 2, 3, or 4 substituent groups as defined herein for the respective groups. Other groups preceded by the prefix “alk-” are defined in the same manner, where “alk” refers to a C₁₋₆ alkylene, unless otherwise noted, and the attached chemical structure is as defined herein.

By “alkcycloalkyl” is meant a cycloalkyl group, as defined herein, attached to the parent molecular group through an alkylene group, as defined herein (e.g., an alkylene group of 1-4, 1-6, or 1-10 carbons). In some embodiments, the alkylene and the cycloalkyl each can be further substituted with 1, 2, 3, or 4 substituent groups as defined herein for the respective group.

By “C₂₋₁₂ alkenyl” or “alkenyl” is meant an optionally substituted unsaturated C₂₋₁₂ hydrocarbon group having one or more carbon-carbon double bonds. Exemplary C₂₋₁₂ alkenyl groups include, but are not limited to —CH═CH (ethenyl), propenyl, 2-propenyl, 2-methyl-1-propenyl, 1-butenyl, 2-butenyl, and the like. A C₂₋₁₂ alkenyl may be linear or branched and may be unsubstituted or substituted. A substituted C₂₋₁₂ alkenyl may have, for example, 1, 2, 3, 4, 5, or 6 substituents located at any position.

The term “alkheteroaryl” refers to a heteroaryl group, as defined herein, attached to the parent molecular group through an alkylene group, as defined herein. In some embodiments, the alkylene and the heteroaryl each can be further substituted with 1, 2, 3, or 4 substituent groups as defined herein for the respective group. Alkheteroaryl groups are a subset of alkheterocyclyl groups.

The term “alkoxy” represents a chemical substituent of formula —OR, where R is a C₁₋₆ alkyl group, unless otherwise specified. In some embodiments, the alkyl group can be further substituted with 1, 2, 3, or 4 substituent groups as defined herein.

By “C₁₋₁₂ alkyl” or “alkyl” is meant an optionally substituted C₁₋₁₂ saturated hydrocarbon group. An alkyl group may be linear, branched, or cyclic (“cycloalkyl”). Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like, which may bear one or more sustitutents. Substituted alkyl groups may have, for example, 1, 2, 3, 4, 5, or 6 substituents located at any position. Exemplary substituted alkyl groups include, but are not limited to, optionally substituted C₁₋₄ alkaryl groups.

The term “alkylene” and the prefix “alk-,” as used herein, represent a saturated divalent hydrocarbon group derived from a straight or branched chain saturated hydrocarbon by the removal of two hydrogen atoms, and is exemplified by methylene, ethylene, isopropylene, and the like. The term “C_{x-y} alkylene” and the prefix “C_{x-y} alk-” represent alkylene groups having between x and y carbons. Exemplary values for x are 1, 2, 3, 4, 5, and 6, and exemplary values for y are 2, 3, 4, 5, 6, 7, 8, 9, or 10. In some embodiments, the alkylene can be further substituted with 1, 2, 3, or 4 substituent groups as defined herein for an alkyl group.

By “C₂₋₁₂ alkynyl” or “alkynyl” is meant an optionally substituted unsaturated C₂₋₆ hydrocarbon group having one or more carbon-carbon triple bonds. Exemplary C₂₋₆ alkynyl groups include, but are not limited to ethynyl, 1-propynyl, and the like.

By “amino” is meant a group having a structure —NR′R″, where each R′ and R″ is selected, independently, from H, optionally substituted C₁₋₆ alkyl, optionally substituted cycloalkyl, optionally substituted heterocyclyl, optionally substituted aryl, optionally substituted heteroaryl, or R′ and R″ combine to form an optionally substituted heterocyclyl. When R′ is not H or R″ is not H, R′ and R″ may be unsubstituted or substituted with, for example, 1, 2, 3, 4, 5, or 6 substituents.

By “aryl” is meant is an optionally substituted C₆-C₁₄ cyclic group with [4n+2] π electrons in conjugation and where n is 1, 2, or 3. Non-limiting examples of aryls include heteroaryls and, for example, benzene, naphthalene, anthracene, and phenanthrene. Aryls also include bi- and tri-cyclic ring systems in which a non-aromatic saturated or partially unsaturated carbocyclic ring (e.g., a cycloalkyl or cycloalkenyl) is fused to an aromatic ring such as benzene or naphthalene. Exemplary aryls fused to a non-aromatic ring include indanyl and tetrahydronaphthyl. Any aryls as defined herein may be unsubstituted or substituted. A substituted aryl may be optionally substituted with, for example, 1, 2, 3, 4, 5, or 6 substituents located at any position of the ring.

The term “aryloxy” represents a chemical substituent of formula —OR, where R is an aryl group of 6 to 18 carbons, as defined herein. In some embodiments, the aryl group can be substituted with 1, 2, 3, or 4 substituents as defined herein. When an aryloxy group is a phenyl group substituted with 1, 2, 3, or 4 halogens, the group is referred to as a “halophenoxy” group.

By “azido” is meant a group having the structure —N₃.

By “carbamate” or “carbamoyl” is meant a group having the structure —OCONR′R″ or —NR′CO₂R″, where each R′ and R″ is selected, independently, from H, optionally substituted C₁₋₆ alkyl, optionally substituted cycloalkyl, optionally substituted heterocyclyl, optionally substituted aryl, optionally substituted heteroaryl, or R′ and R″ combine to form an optionally substituted heterocyclyl. When R′ is not H or R″ is not II, R′ and R″ may be unsubstituted or substituted with, for example, 1, 2, 3, 4, 5, or 6 substituents.

By “carbonate” is meant a group having a the structure —OCO₂R′, where R′ is selected from H, optionally substituted C₁₋₆ alkyl, optionally substituted cycloalkyl, optionally substituted heterocyclyl, optionally substituted aryl, or optionally substituted heteroaryl. When R′ is not H, R may be unsubstituted or substituted with, for example, 1, 2, 3, 4, 5, or 6 substituents.

By “carboxamido” or “amido” is meant a group having the structure —CONR′R″ or —NR′C(═O)R″, where each R′ and R″ is selected, independently, from H, optionally substituted C₁₋₆ alkyl, optionally substituted cycloalkyl, optionally substituted heterocyclyl, optionally substituted aryl, optionally substituted heteroaryl, or R′ and R″ combine to form an optionally substituted heterocyclyl. When R′ is not H or R″ is not H, R′ and R″ may be unsubstituted or substituted with, for example, 1, 2, 3, 4, 5, or 6 substituents.

By “carboxylic ester” is meant a group having a structure selected from —CO₂R′, where R′ is selected from H, optionally substituted C₁₋₆ alkyl, optionally substituted cycloalkyl, optionally substituted heterocyclyl, optionally substituted aryl, or optionally substituted heteroaryl. When R′ is not H, R may be unsubstituted or substituted with, for example, 1, 2, 3, 4, 5, or 6 substituents.

By “carboxylic group” is meant a group having the structure —CO₂R′, where R′ is selected from H, optionally substituted C₁₋₆ alkyl, optionally substituted cycloalkyl, optionally substituted heterocyclyl, optionally substituted aryl, or optionally substituted heteroaryl. When R′ is not H, R may be unsubstituted or substituted with, for example, 1, 2, 3, 4, 5, or 6 substituents.

By “cyano” is meant a group having the structure —CN.

The term “cycloalkyl,” as used herein represents a monovalent saturated or unsaturated non-aromatic cyclic hydrocarbon group of from three to eight carbons, unless otherwise specified, and is exemplified by cyclopropyl, cyclobutyl, cyclopentyl, cyclohexyl, cycloheptyl, bicyclo[2.2.1.]heptyl, and the like.

By “cycloalkenyl” is meant a non-aromatic, optionally substituted 3- to 10-membered monocyclic or bicyclic hydrocarbon ring system having at least one carbon-carbon double bound. For example, a cycloalkenyl may have 1 or 2 carbon-carbon double bonds. Cycloalkenyls may be unsubstituted or substituted. A substituted cycloalkenyl can have, for example, 1, 2, 3, 4, 5, or 6 substituents. Exemplary cycloalkenyls include, but are not limited to, cyclopropenyl, cyclobutenyl, cyclopentenyl, cyclopentadienyl, cyclohexenyl, 1,3-cyclohexadienyl, 1,4-cyclohexadienyl, and the like.

The prefix “halo-” represents a parent molecular group that is substituted by one or more (e.g., 1, 2, 3, 4, or 5) halogen groups, as defined herein.

By “halogen” is meant fluorine (—F), chlorine (—Cl), bromine (—Br), or iodine (—I).

The term “haloalkoxy,” as used herein, represents a group having the structure —OR, where R is a haloalkyl group, as defined herein.

The term “haloalkyl,” as used herein, represents an alkyl group, as defined herein, substituted by a halogen group (i.e., F, Cl, Br, or I). A haloalkyl may be substituted with one, two, three, or, in the case of alkyl groups of two carbons or more, four halogens. Haloalkyl groups include fluoroalkyls (e.g., perfluoroalkyls). In some embodiments, the haloalkyl group can be further substituted with 1, 2, 3, or 4 substituent groups as described herein for alkyl groups.

By “heteroaryl” is mean an aryl group that contains 1, 2, or 3 heteroatoms in the cyclic framework. Exemplary heteroaryls include, but are not limited to, furan, thiophene, pyrrole, thiadiazole (e.g., 1,2,3-thiadiazole or 1,2,4-thiadiazole), oxadiazole (e.g., 1,2,3-oxadiazole or 1,2,5-oxadiazole), oxazole, benzoxazole, isoxazole, isothiazole, pyrazole, thiazole, benzthiazole, triazole (e.g., 1,2,4-triazole or 1,2,3-triazole), benzotriazole, pyridines, pyrimidines, pyrazines, quinoline, isoquinoline, purine, pyrazine, pteridine, triazine (e.g, 1,2,3-triazine, 1,2,4-triazine, or 1,3,5-triazine)indoles, 1,2,4,5-tetrazine, benzo[b]thiophene, benzo[c]thiophene, benzofuran, isobenzofuran, and benzimidazole. Heteroaryls may be unsubstituted or substituted. Substituted heteroaryls can have, for example, 1, 2, 3, 4, 5, or 6 substituents.

By “heterocyclic” or “heterocyclyl” is meant an optionally substituted non-aromatic, partially unsaturated or fully saturated, 3- to 10-membered ring system, which includes single rings of 3 to 8 atoms in size, and polycyclic ring systems (e.g., bi- and tri-cyclic ring systems) which may include an aryl (e.g., phenyl or naphthyl) or heteroaryl group that is fused to a non-aromatic ring (e.g., cycloalkyl, cycloalkenyl, or heterocyclyl), where the ring system contains at least one heterotom. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized or substituted. In certain embodiments, the term heterocylic refers to a non-aromatic 5-, 6-, or 7-membered monocyclic ring wherein at least one ring atom is a heteroatom selected from O, S, and N (wherein the nitrogen and sulfur heteroatoms may be optionally oxidized), and the remaining ring atoms are carbon, the radical being joined to the rest of the molecule via any of the ring atoms. Where a heterocycle is polycyclic, the constituent rings may be fused together, form a spirocyclic structure, or the polycyclic heterocycle may be a bridged heterocycle (e.g., quinuclidyl). Exemplary heterocyclics include, but are not limited to, aziridinyl, azetindinyl, 1,3-diazatidinyl, pyrrolidinyl, piperidinyl, piperazinyl, thiranyl, thietanyl, tetrahydrothiophenyl, dithiolanyl, tetrahydrothiopyranyl, oxiranyl, oxetanyl, tetrahydrofuranyl, tetrahydropyranyl, pyranonyl, 3,4-dihydro-2H-pyranyl, chromenyl, 2H-chromen-2-onyl, chromanyl, dioxanyl (e.g., 1,3-dioxanyl or 1,4-dioxanyl), 1,4-benzodioxanyl, oxazinyl, oxathiolanyl, morpholinyl, thiomorpholinyl, thioxanyl, quinuclidinyl, and also derivatives of said exemplary heterocyclics where the heterocyclic is fused to an aryl (e.g., a benzene ring) or a heteroaryl (e.g., a pyridine or pyrimidine) group. Any of the heterocyclic groups described herein may be unsubstituted or substituted. A substituted heterocycle may have, for example, 1, 2, 3, 4, 5, or 6 substituents.

By “ketone” or “acyl” is meant a group having the structure —COR′, where R′ is selected from H, optionally substituted C₁₋₆ alkyl, optionally substituted cycloalkyl, optionally substituted heterocyclyl, optionally substituted aryl, or optionally substituted heteroaryl. When R′ is not H, R may be unsubstituted or substituted with, for example, 1, 2, 3, 4, 5, or 6 substituents.

By “nitro” is meant a group having the structure —NO₂.

The term “pharmaceutically acceptable solvate” as used herein means an indole compound as described herein wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the molecule is referred to as a “hydrate.”

The term “prodrug,” as used herein, represents compounds that are rapidly transformed in vivo to the parent compound of the above formula, for example, by hydrolysis in blood. Prodrugs of the indole compounds described herein may be conventional esters. Some common esters that have been utilized as prodrugs are phenyl esters, aliphatic (C₁-C₈ or C₈-C₂₄) esters, cholesterol esters, acyloxymethyl esters, carbamates, and amino acid esters. For example, an indole compound that contains an OH group may be acylated at this position in its prodrug form. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins et al., Synthetic Communications 26(23):4351-4367, 1996, each of which is incorporated herein by reference. Preferably, prodrugs of the compounds of the present invention are suitable for use in contact with the tissues of humans and animals with undue toxicity, irritation, allergic response, and the like, commensurate with a reasonable benefit/risk ratio, and effective for their intended use.

By “stereoisomer” is meant a diastereomer, enantiomer, or epimer of a compound. A chiral center in a compound may have the S-configuration or the R-configuration. Enantiomers may also be described by the direction in which they rotate polarized light (i.e., (+) or (−)). Diastereomers of a compound include stereoisomers in which some, but not all, of the chiral centers have the opposite configuration as well as those compounds in which substituents are differently oriented in space (for example, trans versus cis).

By the term “sulfamoyl” is meant a group having a structure according to —NRSO₃R′ or —OSO₂NRR′, where each R or R′ is selected, independently, from H, C₁₋₆ alkyl, C₂₋₆ alkenyl, C₂₋₆ alkynyl, cycloalkyl, heterocyclyl, aryl, or heteroaryl.

The term “thioalkyl,” as used herein, represents a chemical substituent of formula —SR, where R is an alkyl group. In some embodiments, the alkyl group can be further substituted with 1, 2, 3, or 4 substituent groups as described herein.

Where a group is substituted, the group may be substituted with, for example, 1, 2, 3, 4, 5, or 6 substituents. Optional substituents include, but are not limited to: C₁₋₆ alkyl, C₂₋₆ alkenyl, C₂₋₆ alkynyl, cycloalkyl, cycloalkenyl, heterocyclyl, aryl, heteroaryl, halogen; azido(—N₃), nitro (—NO₂), cyano (—CN), acyloxy(—OC(═O)R′), acyl (—C(═O)R′), alkoxy (—OR′), amido (—NR′C(═O)R″ or —C(═O)NRR′), amino (—NRR′), carboxylic acid (—CO₂H), carboxylic ester (—CO₂R′), carbamoyl (—OC(═O)NR′R″ or —NRC(═O)OR′), hydroxy (—OH), isocyanato (—NC), sulfonate (—S(═O)₂OR), sulfonamide (—S(═O)₂NRR′ or —NRS(═O)₂R′), or sulfonyl (—S(═O)₂R), where each R or R′ is selected, independently, from 1-1, C₁₋₆ alkyl, C₂₋₆ alkenyl, C₂₋₆ alkynyl, cycloalkyl, heterocyclyl, aryl, or heteroaryl. A substituted group may have, for example, 1, 2, 3, 4, 5, 6, 7, 8, or 9 substituents. In some embodiments, each hydrogen in a group may be replaced by a substituent group (e.g., perhaloalkyl groups such as —CF₃ or —CF₂CF₃ or perhaloaryls such as —C₆F₅). In other embodiments, a substituent group may itself be further substituted by replacing a hydrogen of said substituent group with another substituent group such as those described herein. Substituents may be further substituted with, for example, 1, 2, 3, 4, 5, or 6 substituents as defined herein. For example, a lower C₁₋₆ alkyl or an aryl substituent group (e.g., heteroaryl, phenyl, or naphthyl) may be further substituted with 1, 2, 3, 4, 5, or 6 substituents as described herein.

By “preventing” or “reducing the likelihood of” is meant reducing the severity, the frequency, and/or the duration of a condition or disorder or the symptoms thereof. For example, reducing the likelihood of or preventing a condition or disorder associated with muscular dysfunction is synonymous with prophylaxis or the chronic treatment of the condition or disorder.

By “reducing muscle fatigue” is meant a decrease in the rate of muscle fatigue when a muscle is repetitively stimulated, resulting in an increased ability to, for example, lift objects, move about, participate in or maintain participation in physical activity (e.g., walking or other exercise) with a reduction in muscle fatigue. A reduction in muscle fatigue may also result in enhanced endurance.

By “sarcopenia” is meant a decrease in muscle mass in a subject due to aging, resulting in a decrease in muscle strength and/or increase in muscle fatigue.

By “skeletal muscle” is meant skeletal muscle tissue as well as components thereof, such as skeletal muscle fibers (i.e., fast or slow skeletal muscle fibers), connective tissue, vasculature, nerve supply the myofibrils comprising the skeletal muscle fibers, the skeletal sarcomere which comprises the myofibrils, and the various components of the skeletal sarcomere.

By “subject” is meant any animal, e.g., a mammal (e.g., a human). Other animals that can be treated using the compositions and methods of the invention include, e.g., horses, dogs, cats, pigs, goats, rabbits, hamsters, monkeys, guinea pigs, rats, mice, lizards, snakes, sheep, cattle, fish, and birds. A subject who is being treated for muscular dysfunction is one who has been diagnosed by a medical practitioner as having such a condition. One in the art will understand that subjects of the invention may have been subjected to standard tests or may have been identified, without examination, as one at high risk due to the presence of one or more risk factors, such as age, genetics, or family history.

By “sustained release” or “controlled release” is meant that the therapeutically active component is released from the formulation at a controlled rate such that therapeutically beneficial blood levels (but below toxic levels) of the component are maintained over an extended period of time ranging from, e.g., about 12 to about 24 hours, thus, providing, for example, a 12-hour or a 24-hour dosage form.

By “systemic administration” is meant any non-dermal route of administration, and specifically excludes topical and transdermal routes of administration.

By “therapeutic agent” is meant any agent that produces a healing, curative, stabilizing, or ameliorative effect.

By “treating or ameliorating” is meant ameliorating a condition or symptoms of the condition before or after its onset. As compared with an equivalent untreated control, such amelioration or degree of treatment is at least 5%, 10%, 20%, 30%, 40%, 50%, 60%, 70%, 80%, 90%, 95%, or 100%, as measured by any standard technique.

Other features and advantages of the invention will be apparent from the detailed description and from the claims.

### BRIEF DESCRIPTION OF THE DRAWINGS

FIGS. 1A-1D are schematic representations illustrating the physiological screening technology (MyoForce Analysis System (MFAS™)) used in the Examples described herein. The schematic highlights the tissue culture components (FIG. 1A), miniature BioArtificial Muscle (mBAM) formation (top view and side view; FIG. 1B), muscle differentiation into contractile muscle fibers (stained for sarcomeric tropomyosin; FIG. 1C), and development of muscle force generation with differentiation and plateau reached after 7-9 days in culture (FIG. 1D). Test drugs are added, and change in force is measured over the next 3-4 days. Scale bars, 6 mm (FIG. 1A); 6 mm and 3 mm (FIG. 1B). See, e.g., Vandenburgh, Tissue Eng Part B Rev. 16: 55-64, 2010.

FIGS. 2A and 2B are schematic representations of the high content MFAS™ assay method. Muscle forces are measured via a mechanical model that correlates post displacement with force (FIG. 2A) collected with a custom imaging system (FIG. 2B) that coverts micrometers of movement to micronewtons of force and results from the sum effects of a drug on the multiple biochemical pathways affecting muscle strength.

FIG. 3 is a graph showing the effect of metformin on mdx murine muscle tissue strength. Statistical analysis of experimental data versus no-drug controls was performed using t-tests. The black bar on the x-axis is the reported range of metformin plasma concentrations in human diabetic patients on metformin therapy (Marchetti et al., Clin Pharm Ther. 41: 450-454, 1987). (Mean±SEM of mBAM maximal tetanic forces after four days of exposure to metformin.)

FIGS. 4A and 4B are graphs showing the effect of low concentrations of metformin on mdx murine skeletal muscle tissue strength (FIG. 4A) and fatigue rate (FIG. 4B). Statistical analysis of experimental data versus no-drug controls was performed using t-tests. The black bar on the x-axis of each graph is the reported range of metformin plasma concentrations in human diabetic patients on metformin therapy. (Mean±SEM of mBAM maximal tetanic forces after four days of exposure to metformin.)

FIGS. 5A-5C are graphs showing the effect of biguanides (metformin, FIG. 5A; phenformin, FIG. 5B; and proguanil, FIG. 5C) on muscle strength in normal marine skeletal muscle tissue. Statistical analysis of experimental data versus no-drug controls was performed using t-tests. The black bar on the x-axis of each graph is the reported range of plasma concentrations of the biguanide in human diabetic patients on biguanide therapy (metformin, FIG. 5A and phenformin, FIG. 5B) or in patients on anti-malarial medication (proguanil, FIG. 5C). (Mean±SEM of mBAM tetanic forces after three to four days of exposure to varying doses of a biguanide.)

FIGS. 6A-6D are graphs showing the effect of biguanides (metformin, FIG. 6A; phenformin, FIG. 6B; buformin, FIG. 6C; and proguanil, FIG. 6D) on the rate of fatigue of normal mouse skeletal muscle tissue. Statistical analysis of experimental data versus no-drug controls was performed using t-tests. (Mean±SEM of mBAM tetanic forces after three to four days of exposure to a biguanide.)

FIGS. 7A-7D are graphs showing the effect of biguanides (metformin, FIG. 7A; phenformin, FIG. 7B; buformin, FIG. 7C; and proguanil, FIG. 7D) on muscle strength in normal human muscle tissue. Statistical analysis of experimental data versus no-drug controls was performed using t-tests. The black bar on the x-axis of each graph is the reported range of plasma concentrations of the biguanide in human diabetic patients (metformin, FIG. 7A; phenformin, FIG. 7B; and buformin, FIG. 7C) or in patients on anti-malarial medication (proguanil; FIG. 7D). (Mean±SEM of mBAM tetanic forces after three to four days of exposure to varying doses of a biguanide.)

FIGS. 8A-8D are graphs showing the effect of biguanides (metformin, FIG. 8A; phenformin, FIG. 8B; buformin, FIG. 8C; and proguanil, FIG. 8D) on the rate of fatigue of normal human muscle tissue. Statistical analysis of experimental data versus no-drug controls was performed using t-tests. (Mean±SEM of mBAM tetanic forces after three to four days of exposure to a biguanide.)

FIGS. 9A-9D are graphs showing the effect of biguanides (metformin, FIG. 9A; phenformin, FIG. 9B; buformin, FIG. 9C; and proguanil, FIG. 9D) on muscle strength of human Duchenne muscular dystrophy (DMD) muscle tissue. Statistical analysis of experimental data versus no-drug controls was performed using t-tests. The black bar on the x-axis of each graph is the reported range of biguanide plasma concentrations in human diabetic patients. (Mean±SEM of mBAM tetanic forces after three to four days of exposure to varying concentrations of a biguanide.) FIGS. 10A-10C are graphs showing the effect of biguanides (metformin, FIG. 10A; phenformin, FIG. 10B; and buformin, FIG. 10C) on the rate of muscle fatigue of human DMD muscle tissue. Statistical analysis of experimental data versus no-drug controls was performed using t-tests. (Mean±SEM of mBAM tetanic forces after three to four days of exposure to a biguanide.)

### DETAILED DESCRIPTION

We have discovered that low dosages of biguanides, commonly used to treat diabetes, increase muscle strength and reduce muscle fatigue in rodent and human muscle tissue. Thus, the methods of the present invention may be useful in treating muscular dysfunction associated with a condition or disorder, such as, e.g., muscular dystrophy, sarcopenia, cachexia, cancer, acquired immune deficiency syndrome, advanced organ failure, chronic obstructive pulmonary disease, rhabdomyolysis, disuse atrophy, incontinence, sepsis, neuromuscular disease, and congenital myopathy. Additionally, the methods of the invention may be used to increase muscle strength, muscle mass, or muscle endurance and decrease muscle fatigue in a subject. Administration of low dosages of a biguanide may also be used to treat or prevent cancer and may be used to expand the lifespan of a subject.

### Biguanides

Biguanides or pharmaceutically acceptable salts thereof may be used in the methods and compositions of the present invention. Biguanides have a chemical structure shown in formula (I), above, which is based on the following structure:

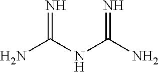

Exemplary biguanides are provided in Table 1.

TABLE 1
Exemplary biguanides

| | |
|---|---|
| Metformin (1,1- dimethylimido- dicarbonimidic diamide) | 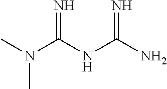 |
| Phenformin | 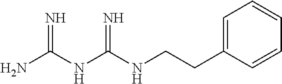 [SMILES: N=C(N)NC(=N)NCCc1ccccc1] |
| Buformin | 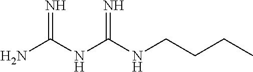 |
| Proguanil (1-(4- chlorophenyl)-5- isopropyl- biguanide) | 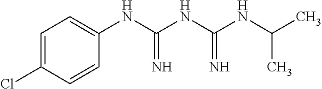 [SMILES: CC(C)NC(=N)NC(=N)Nc1ccc(Cl)cc1] |

Additional biguanides are described, for example, in U.S. Pat. Nos. 2,371,111; 2,961,377; 2,990,425; 3,057,780; 3,174,901; 3,222,398; 3,800,043; 3,821,406; 3,830,933; 3,959,488; 3,996,232; 4,017,539; 4,028,402; 4,080,472; 5,286,905; 5,376,686; 5,955,106; 6,031,004; 7,199,159; 7,256,218; 7,285,681; 7,396,858; and 7,563,792; U.S. Patent Application Publication Nos. 2003/0187036; 2003/0220301; 2004/0092495; 2004/0116428; 2005/0124693; 2005/0182029; and 2008/0176852, hereby incorporated by reference.

### Diagnosis and Treatment

Methods of the invention include administering to a subject a therapeutically effective amount of a biguanide or a pharmaceutically acceptable salt thereof (e.g., a low dosage of a biguanide or a pharmaceutically acceptable salt thereof) to treat muscular dysfunction, to increase muscle strength, or to decrease muscle fatigue in a subject.

In one embodiment, the methods of the present invention are used to treat muscular dysfunction. Conditions and disorders associated with muscular dysfunction include, without limitation, muscular dystrophy (e.g., Duchenne, Becker, limb girdle, congenital, facioscapulohumeral, myotonic, oculopharyngeal, distal, spinal, or Emery-Dreifuss muscular dystrophy), Brown-Vialetto-Van Laere syndrome, Fazio-Londe syndrome, Lambert-Eaton myasthenic syndrome, cancer, acquired immune deficiency syndrome (AIDS), advanced organ failure (e.g., heart, liver, or kidney failure), chronic obstructive pulmonary disease (COPD), rhabdomyolysis, tissue hypoxia (e.g., peripheral claudication and exercise intolerance in diabetic subjects), angina, myocardial infarction, disuse atrophy due to prolonged immobility (e.g., resulting from solid organ transplant, joint replacement, stroke, spinal cord injury, recovery from severe burn, or sedentary chronic hemodialysis), Dejerine Sottas syndrome, incontinence, sepsis, aging, neuromuscular diseases (e.g., stroke, Parkinson's disease, multiple sclerosis, myasthenia gravis, Huntington's disease (e.g., Huntington's chorea), or Creutzfeldt-Jakob disease), amyotrophic lateral sclerosis (ALS), post-polio muscular atrophy, chronic muscle fatigue syndrome, or congenital myopathies. Symptoms of muscular dysfunction include, e.g., progressive muscular wasting, low muscle mass, poor balance, frequent falls, walking difficulty, low gait speed, waddling gait, calf deformation, limited range of movement, respiratory difficulty, drooping eyelids, scoliosis, or the inability to walk or lift objects. One skilled in the art will understand that subjects of the invention may have been subjected to standard tests or may have been identified, without examination, as one at high risk due to the presence of one or more risk factors. Diagnosis of these disorders may be performed using any standard method known in the art.

In certain embodiments, the compositions and methods of the invention may be used to counter muscle fatigue and weakness in a subject that is being treated with one or more statins (e.g., atorvastatin, rosuvastatin, lovastatin simvastatin, pravastatin, cerivastatin, or fluvastatin).

In other embodiments, the compositions and methods of the invention may be used to increase muscle strength and/or decrease muscle fatigue in a subject that, for example, may or may not be experiencing muscular dysfunction. In certain embodiments, the methods provided herein may be used to improve athletic performance. For example, the methods may be used to shorten the time normally needed to recover from physical exertion or to increase muscle strength of a subject (e.g., an athlete engaged in a professional or recreational sport or activity, including, but not limited to, weight-lifting, body-building, track and field events, and any of various team sports).

In some embodiments, methods of the invention may be used to treat or prevent cancer, and low dosages of a biguanide (alone or in combination with other therapeutic regimen) may be used to extend the lifespan of a subject.

The efficacy of treatment can be monitored using methods known to one of skill in the art including, e.g., assessing symptoms of a disease or disorder, physical examination, histopathological examination (e.g., muscle biopsy), genetic testing, blood chemistry analysis (e.g., measuring the level of creatine kinase in the blood), computed tomography, cytological examination, and magnetic resonance imaging. Muscle strength measurements may include, but are not limited to, hand-held dynamometry measurements, maximum voluntary contraction (MVC) strain gauge measurements, spirometry, and manual muscle testing. Muscle strength tests may use an instrument that measures how much force (for example, pounds of force) an individual can apply to the instrument using a selected group of muscles, such as, e.g., the hand muscles. Other methods and devices for evaluating muscle function, muscle strength, and/or muscle endurance are found, for example, in U.S. Pat. Nos. 5,263,490; 6,546,278; and 7,470,233, and in U.S. Patent Application Publication Nos. 2007/0129771 and 2009/0227906.

The methods of the present invention may be used in combination with additional therapies in the methods described herein. Therapies that can be used in combination with the methods of the invention include, but are not limited to, the administration of additional therapeutic agents (e.g., mexiletine, phenyloin, baclofen, dantrolene, carbamazepine, muscle relaxants (e.g., cyclobenzaprine or tizanidine), glucocorticoids (e.g., prednisone or deflazacort), chemotherapeutic agents, anti-inflammatory agents, β-hydroxy β-methylbutyrate, protein and amino acid supplements, creatine, carnitine, taurine, multi-vitamins and minerals, anti-estrogenic compounds, or herbal supplements), surgical intervention, or behavioral therapies (e.g., exercise or physical therapy).

### Formulation

Any of the therapeutic agents employed according to the present invention may be contained in any appropriate amount in any suitable carrier substance, and such therapeutic agents are generally present in an amount of 1-95% by weight of the total weight of the composition. The composition may be provided in a dosage form that is, e.g., suitable for topical, oral, subcutaneous, intravenous, intracerebral, intranasal, transdermal, intraperitoneal, intramuscular, intrapulmonary, rectal, intra-arterial, intralesional, parenteral, or intra-ocular administration. Accordingly, the composition may be in the form of, e.g., tablets, capsules, pills, powders, granulates, suspensions, emulsions, solutions, gels (e.g., hydrogels), pastes, ointments, creams, plasters, drenches, osmotic delivery devices, suppositories, enemas, injectables, implants, sprays, or aerosols. For example, the therapeutic agent may be in the form of a pill, tablet, capsule, liquid, or sustained release tablet for oral administration; a liquid for intravenous administration, subcutaneous administration, or injection; a powder, nasal drop, or aerosol for intranasal administration; or a polymer or other sustained-release vehicle for local administration. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Encapsulation of the therapeutic agent in a suitable delivery vehicle (e.g., polymeric microparticles or implantable devices) may increase the efficiency of delivery, particularly for oral delivery.

If more than one therapeutic agent is employed, each agent may be formulated separately or together using methods known in the art. In one embodiment, the agents are formulated together for the simultaneous or near simultaneous administration of the agents. Such co-formulated compositions can include the two agents formulated together in the same, e.g., pill, capsule, or liquid.

The therapeutic agent(s) may also be packaged as a kit. Non-limiting examples include kits that contain, e.g., two pills, a pill and a powder, a suppository and a liquid in a vial, or two topical creams. The kit can include optional components that aid in the administration of the unit dose to patients, such as vials for reconstituting powder forms, syringes for injection, customized intravenous delivery systems, or inhalers. Additionally, the unit dose kit can contain instructions for preparation and administration of the compositions. The kit may be, e.g., manufactured as a single use unit dose for one patient, multiple uses for a particular patient (e.g., at a constant dose or in which the individual compounds may vary in potency as therapy progresses), or the kit may contain multiple doses suitable for administration to multiple patients (e.g., bulk packaging). The kit components may be assembled in, e.g., cartons, blister packs, bottles, or tubes.

### Dosages and Administration

Generally, when administered to a human subject, the dosage of any of the agents of the invention (e.g., biguanides) will depend on the nature of the agent and can readily be determined by one skilled in the art. Typically, such dosage is normally about 0.001 mg to 2000 mg per day, preferably less than 500, 400, 300, 200, 100, 90, 80, 70, 60, 50, 40, 30, 20, 10, 5, 1, 0.5, 0.1, or 0.01 mg per day. Appropriate dosages of compounds used in the methods described herein depend on several factors, including the administration method, the severity of the disorder, and the age, weight, and health of the subject to be treated. Additionally, pharmacogenomic information (e.g., the effect of genotype on the pharmacokinetic, pharmacodynamic, or efficacy profile of a therapeutic) about a particular subject may affect the dosage used.

In certain embodiments, the therapeutically effective amount of a biguanide administered to a subject results in a concentration between about 0.0000001 μg/ml to about 10.0 μg/ml, about 0.0000001 μg/ml to about 1.0 μg/ml, about 0.0000001 μg/ml to about 0.1 μg/ml, about 0.0000001 μg/ml to about 0.01 μg/ml, about 0.0000001 μg/ml to about 0.001 μg/ml, about 0.0000001 μg/ml to about 0.0001 μg/ml, about 0.0000001 μg/ml to about 0.00001 μg/ml, or about 0.0000001 μg/ml to about 0.000001 μg/ml in blood, serum, or plasma of the subject.

In certain embodiments, pharmaceutical compositions of the invention are formulated to include a biguanide or pharmaceutically acceptable salt thereof at a dosage unit of less than 250 milligrams. For example, the composition may include as little as 0.0001, 0.001, 0.01, 0.05, 0.1, 0.5, 1, 2, 3, 4, 5, or 10 mg of a biguanide or as much as 20, 40, 60, 80, 120, 140, 160, 180, 220, or 240 mg of a biguanide.

Administration of a biguanide, alone or in combination with another therapeutic agent, can be one to four times daily for one day to one year and may even be for the life of the subject. Depending upon the half-life of the biguanide in a particular subject, the therapeutic agent may be administered several times per day to once a week, once a month, or once a year. Alternatively, a single administration may be satisfactory. Thus, the methods described herein provide for a single administration as well as multiple administrations that are given either simultaneously or over an extended period of time.

### EXAMPLES

The present invention is illustrated by the following examples, which are in no way intended to be limiting of the invention.

### Example 1

### Metformin Increases Strength and Reduces the Fatigue Rate of Mouse Skeletal Muscle Tissue from a Genetic Model of Duchenne Muscular Dystrophy

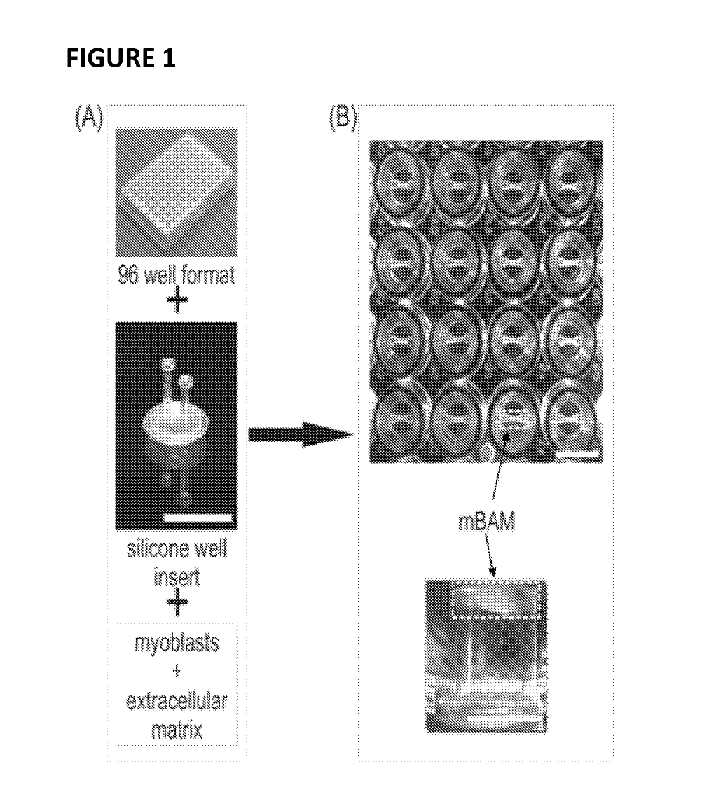

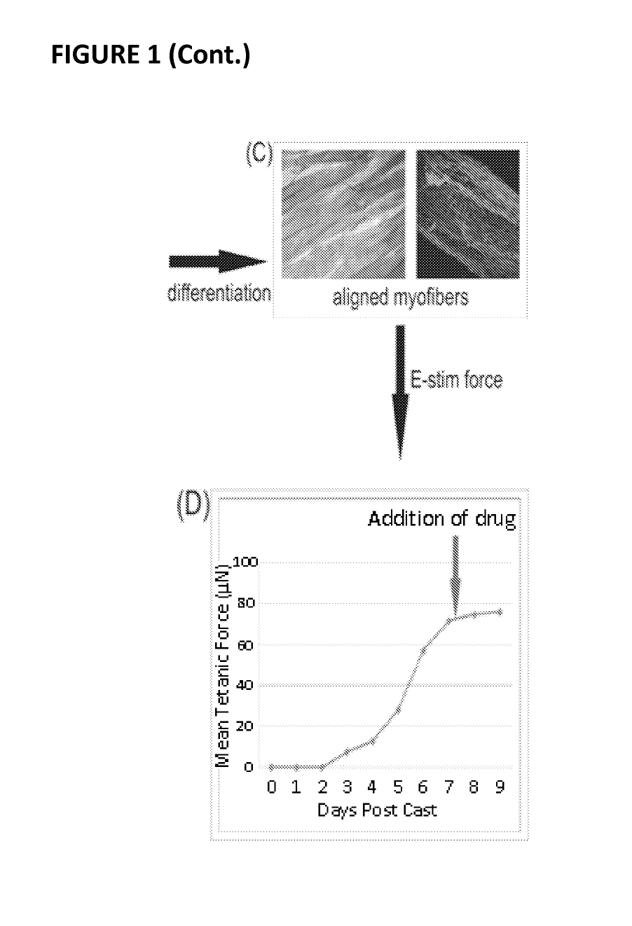

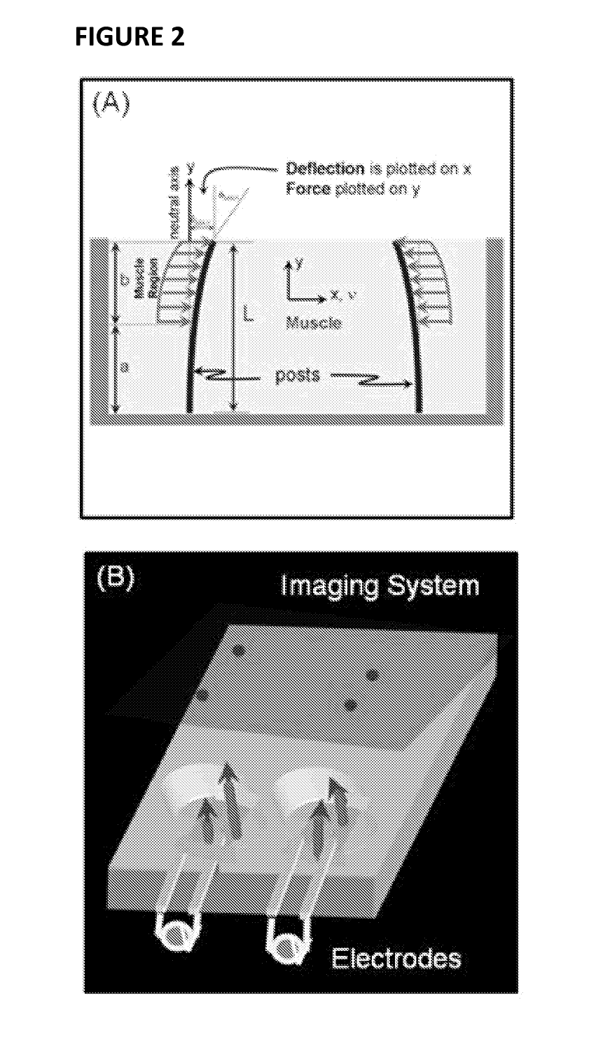

Conditionally immortalized skeletal muscle cells from the mdx mouse muscle (Morgan et al., Dev Biol. 162: 486-498, 1994) were tissue engineered into contractile muscle tissue (mBAMs), as described herein. (See also, e.g., Vandenburgh et al., Muscle and Nerve 37: 438-447, 2008; Vandenburgh et al., Treat NMD Meeting, 2009). Briefly, several hundred thousand proliferating myoblasts were mixed with a solution of extracellular matrix (collagen or fibrin) and pipetted into custom wells containing two flexible “posts” (FIG. 1A). The cell/matrix mixture gelled around the tops of the posts, forming a defined tubular structure (FIG. 1B). The myoblasts differentiated into several hundred organized contractile muscle fibers (called miniature BioArtificial Muscle (mBAM); FIG. 1C) in tissue culture medium, such as that described in Vandenburgh et al., FASEB J. 23: 3325-3334, 2009. After 1-2 weeks in tissue culture, mBAMs generated a constant level of maximal tetanic force when electrically stimulated (FIG. 1D). Maximal tetanic force (i.e., muscle strength) was measured by imaging the deflection of the attachment posts and converting the distance of deflection into force, as illustrated in FIGS. 2A-2B.

High glucose tissue culture medium (4.5 g/l) was used in all experiments to maintain glucose levels well above normal human blood plasma levels (0.9 to 1.8 g/l) and, thereby, minimize drug action through increased glucose availability to the muscle cells. Varying concentrations of metformin (Sigma-Aldrich Cat. No. D15,095-9; 1,1-dimethylbiguanide hydrochloride) were added to tissue culture medium of mBAMs when the mBAMs had reached a plateau of maximal tetanic force, which was measured in a MyoForce Analysis System (MFAS™) 24 hours later. Every 24 hours, fresh metformin was added to fresh tissue culture medium, and tetanic forces were measured every 24 hours for 3-4 days. Fatigue was assayed after 3-4 days of metformin treatment by 15-20 repetitive tetanic stimulations of each mBAM at 14V, 60 Hz for 2 seconds every 4-5 seconds. Force is determined after each stimulation and compared to the initial force determined after the first stimulation relative to untreated controls. (Mean±S.E.M. of 3-8 samples per group was calculated and statistical analyses performed by t-tests.)

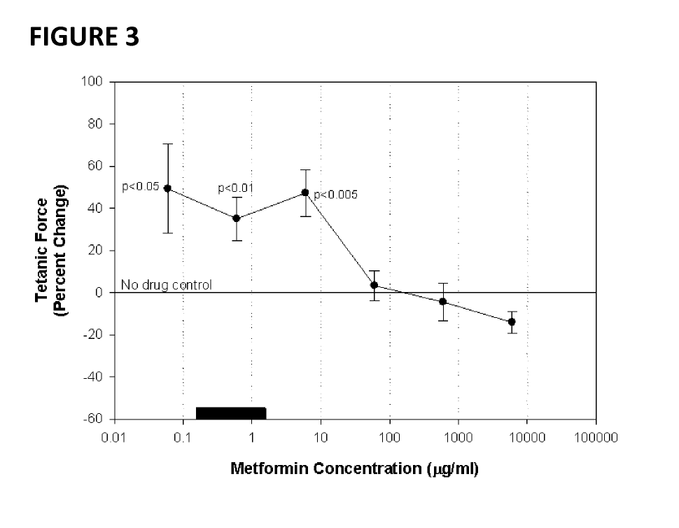

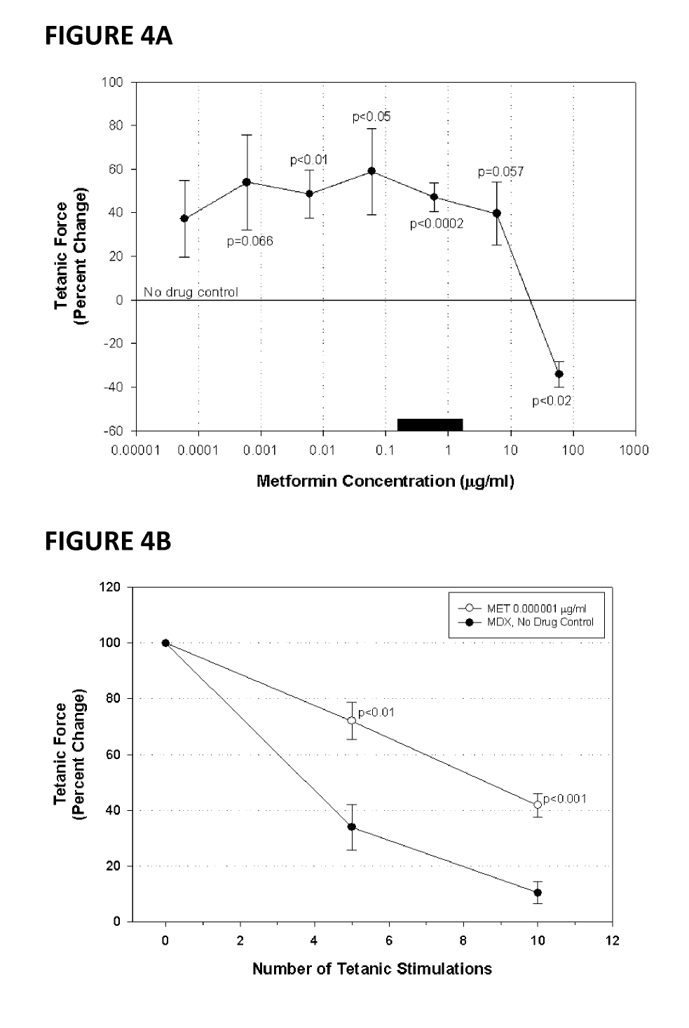

Metformin concentrations between 0.06 μg/ml and 6 μg/ml significantly increased maximal tetanic force generated by mBAM muscle tissue compared to untreated control muscle tissue (FIG. 3). A metformin concentration of 0.0006 μg/ml significantly increased maximal tetanic force generated by mBAM muscle tissue (FIG. 4A). Additionally, metformin at a concentration of 0.000001 μg/ml significantly decreased the rate of muscle fatigue (FIG. 4B). The results of these experiments indicate that metformin increases muscle strength and decreases muscle fatigue of dystrophic mouse muscle tissue at concentrations at, or less than, mean plasma levels in subjects taking a biguanide for other disease indications.

### Example 2

### Biguanides Increase Skeletal Muscle Strength and Decrease the Rate of Muscle Fatigue of Normal Mouse Skeletal Muscle Tissue

Skeletal muscle cells from the leg muscles of normal mice were isolated by standard tissue culture protocols (Rando et al., J Cell Biol. 125: 1275-1287, 1994), tissue engineered into contractile muscle tissue (mBAMs), and muscle force and fatigue measured as described in Example 1 (above).

High glucose tissue culture medium (4.5 g/l) was used in all experiments to maintain glucose levels well above normal human blood plasma levels (0.9 to 1.8 g/l) and, thereby, minimize drug action through increased glucose availability to the muscle cells. On Days 6-8 post-casting, metformin, phenformin (Fluka Cat. No. P7045-1G), or proguanil (Ipca Laboratories, CAS No. 637-32-1, Batch No. 8001HPRI) at different concentrations were added to the tissue culture medium, and maximal tetanic force was measured 24 hours later in a MFAS™. Every 24 hours, fresh metformin, phenformin, or proguanil was added in fresh medium and tetanic force measured every 24 hours for 3-4 days, as described in Example 1.

In a separate experiment, metformin, phenformin, buformin (Santa Cruz Biotech Cat. No. SC-207383), or proguanil were added to the tissue culture medium daily for 3-4 days, and the rate of muscle fatigue was determined by following the rate of reduction in maximal tetanic force when the muscle tissue was electrically stimulated repetitively, as described in Example 1.

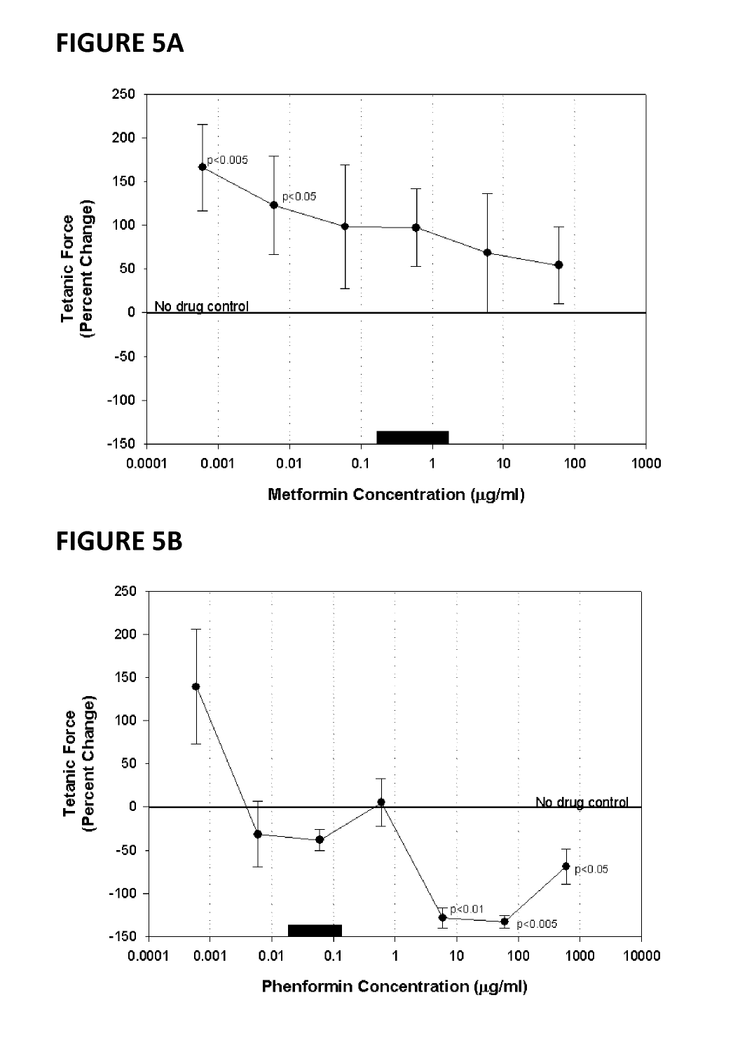

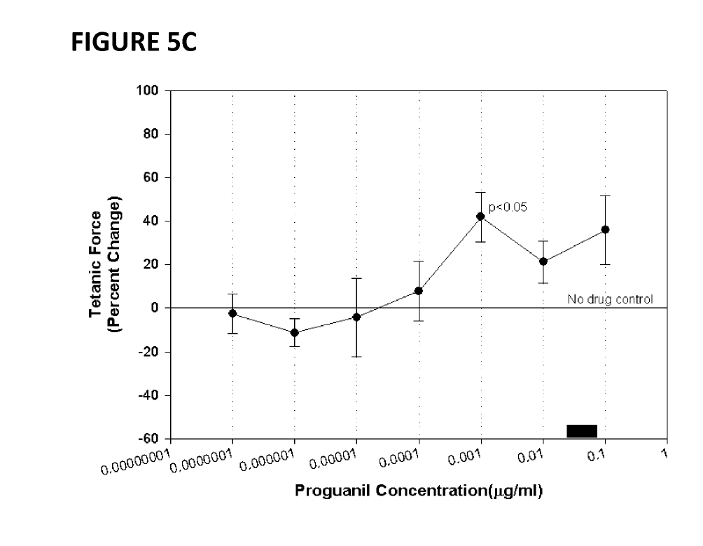

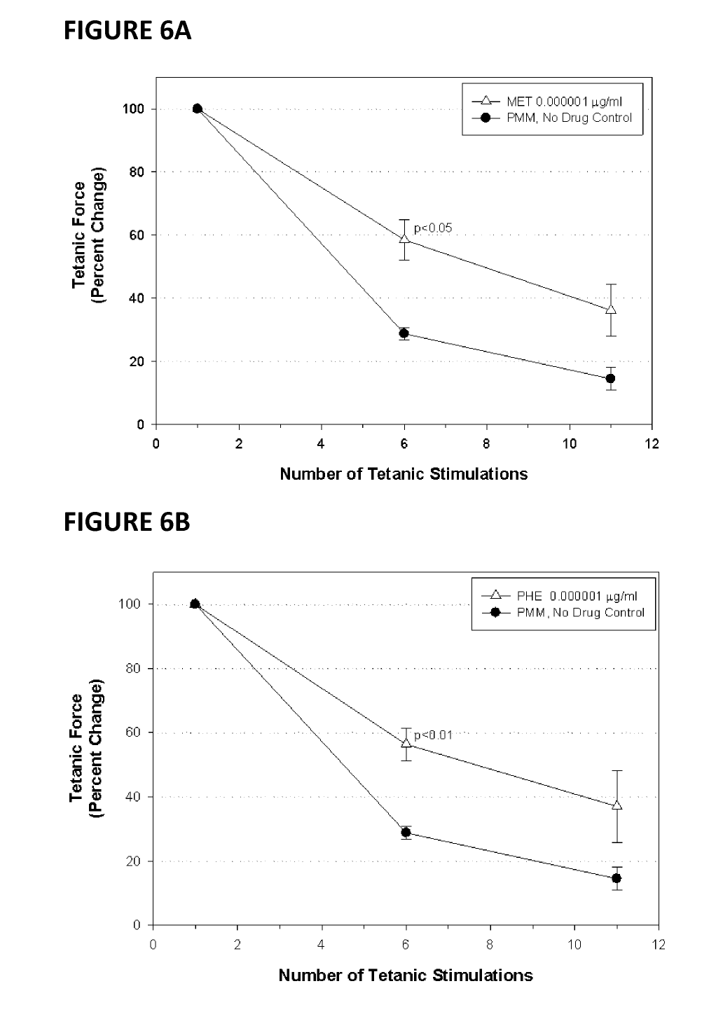

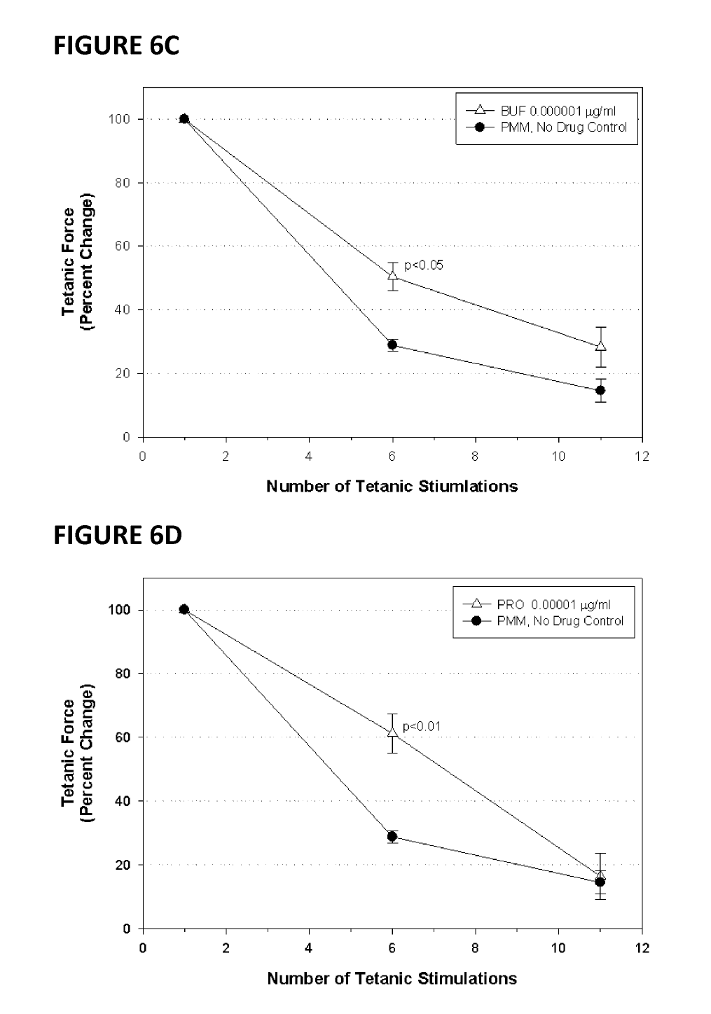

A metformin concentration of 0.0006 μg/ml significantly increased tetanic force generated by the muscle tissue (FIG. 5A). High concentrations of phenformin (>5 μg/ml) were toxic to muscle tissue, while low concentrations (<0.001 μg/ml) increased tetanic force (FIG. 5B). Proguanil (0.001 μg/ml) significantly increased tetanic force after 3 days of treatment (FIG. 5C). Low concentrations of all biguanides significantly decreased the rate of muscle fatigue (FIGS. 6A-6D).

The results of these experiments indicate that biguanides increase skeletal muscle strength and reduce the rate of muscle fatigue in normal mouse skeletal muscle tissue at concentrations at, or less than, mean plasma levels in subjects taking a biguanide for other disease indications.

### Example 3

### Biguanides Increase Skeletal Muscle Strength and Reduce the Rate of Muscle Fatigue of Normal Human Muscle Tissue

Skeletal muscle cells from the vastus lateralis muscle of human volunteers were isolated by thin needle muscle biopsy and grown by standard tissue culture protocols (Shansky et al., “Tissue engineering human skeletal muscle for clinical applications” in Culture of Cells for Tissue Engineering (G. Vunjak and I. Freshney, eds.), pages 239-257, 2006), tissue engineered into contractile muscle tissue (mBAMs), and muscle force and fatigue measured as described in Example 1. On Days 7-10 post-casting, metformin, phenformin, buformin, or proguanil at different concentrations were added to the tissue culture medium and maximal tetanic force measured 24 hours later in MFAS™. High glucose tissue culture medium (4.5 g/l) was used to maintain glucose levels well above normal human blood plasma levels (0.9 to 1.8 g/l) and, thereby, minimize drug action through increased glucose availability to the muscle cells. Every 24 hours, fresh metformin, phenformin, buformin, or proguanil was added in fresh tissue culture medium and maximal tetanic force measured every 24 hours for a total of 3-4 days. At the end of 3-4 days of drug treatment, the rate of muscle fatigue was determined by rapid repetitive tetanic stimulation of the muscle tissue, as described above.

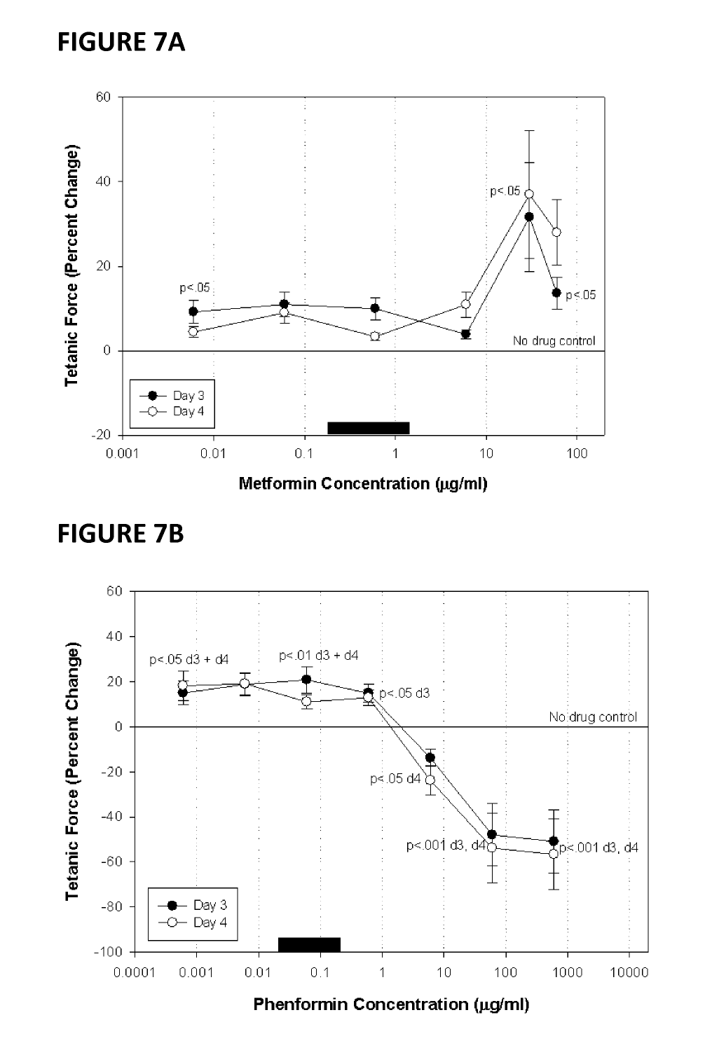

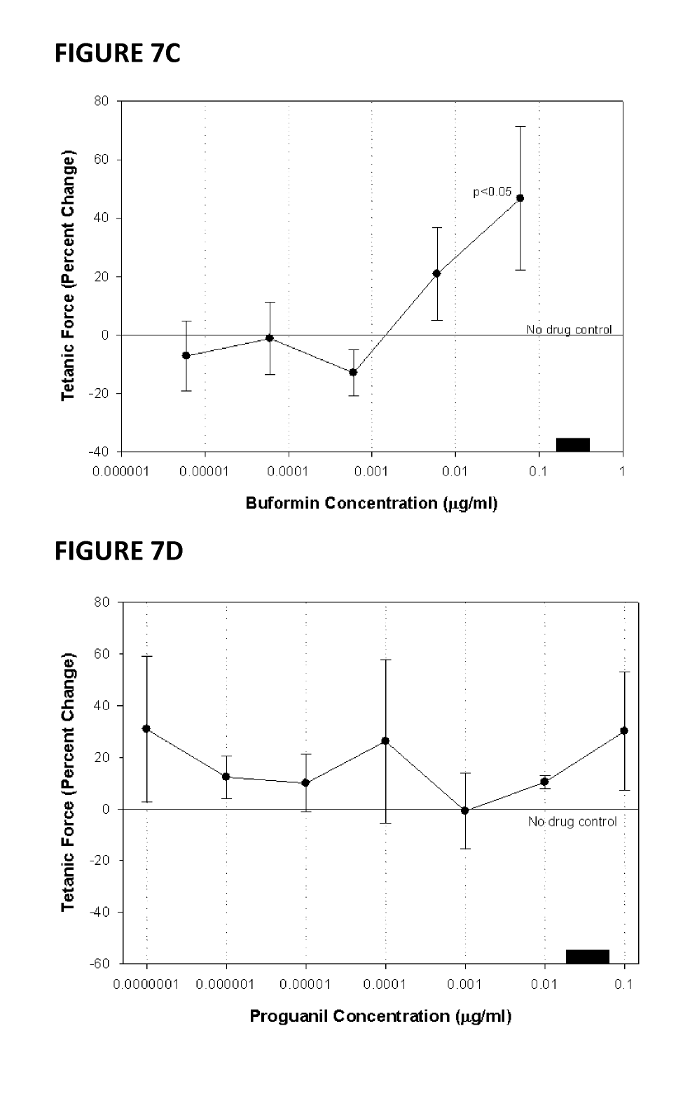

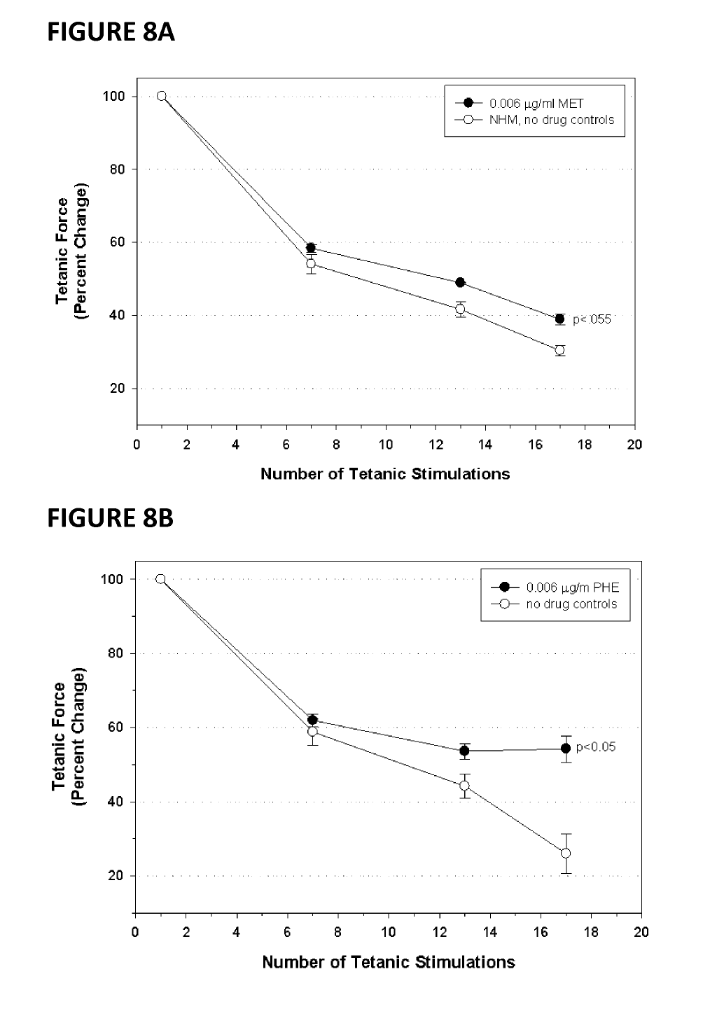

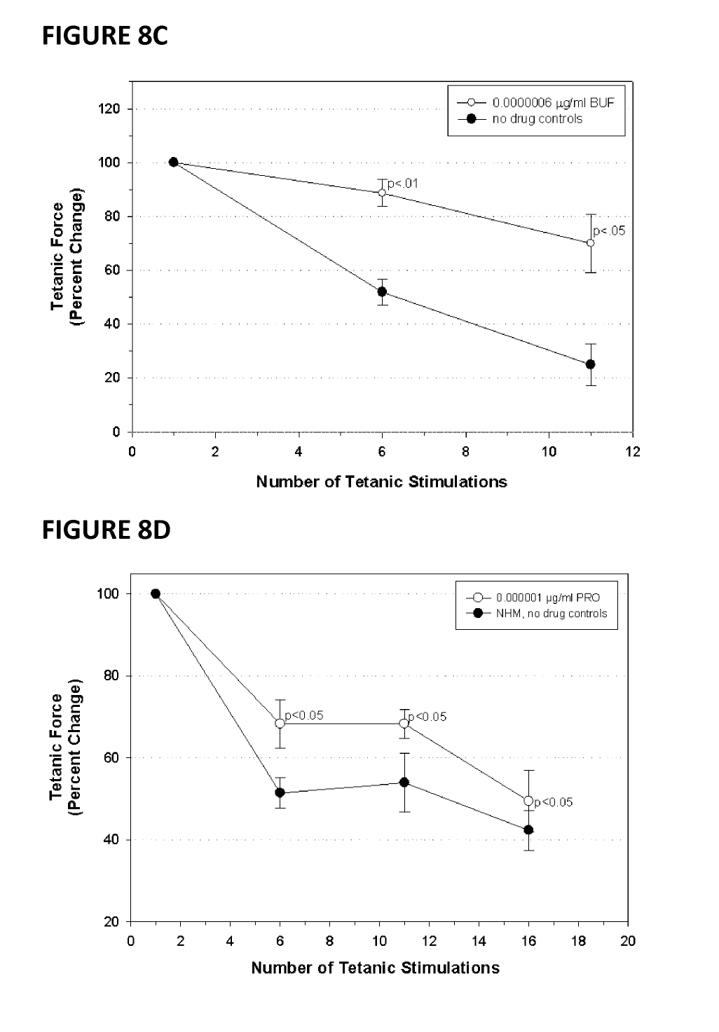

Metformin concentrations between 0.006 μg/ml and 0.06 μg/ml significantly increased tetanic force generated by the muscle tissue (FIG. 7A). High concentrations of phenformin (>5 μg/ml) were toxic to the muscle tissue, while low concentrations (<0.001 μg/ml) increased tetanic force (FIG. 7B). Concentrations of buformin less than 0.1 μg/ml increased muscle tissue tetanic force (FIG. 7C). Concentrations of proguanil less than 0.0001 μg/ml increased muscle tissue tetanic force (FIG. 7D). Similar results were obtained for the biguanides using muscle tissue generated from cells isolated from different muscle biopsies (data not shown). Low concentrations of all four biguanides also decreased the rate of muscle fatigue with repetitive stimulations (FIGS. 8A-8D).

The results of these experiments indicate that biguanides increase skeletal muscle strength and reduce the rate of muscle fatigue in normal human skeletal muscle tissue at concentrations at, or less than, mean plasma levels in subjects taking a biguanide for other disease indications.

### Example 4

### Biguanides Increase Skeletal Muscle Strength and Reduce the Rate of Muscle Fatigue of Skeletal Muscle Tissue from a Subject with Duchenne Muscular Dystrophy

Skeletal muscle cells from a subject with Duchenne muscular dystrophy were isolated, and tissue culture protocols were used to immortalize the muscle cells (Zhu et al., Aging Cell 6: 515-523, 2007). The immortalized muscle cells were tissue engineered into contractile muscle tissue (mBAMs), and muscle force and fatigue were measured as described above. High glucose tissue culture medium (4.5 g/l) was used to maintain glucose levels well above normal human blood plasma levels (0.9 to 1.8 g/l) and, thereby, minimize drug action through increased glucose availability to the muscle cells. On Days 7-10 post-casting, metformin, phenformin, buformin, or proguanil at different concentrations was added to the tissue culture medium and maximal tetanic force measured 24 hours later in MFAS™. Every 24 hours, fresh biguanide was added in fresh medium and tetanic force measured for a total of 3-4 days.

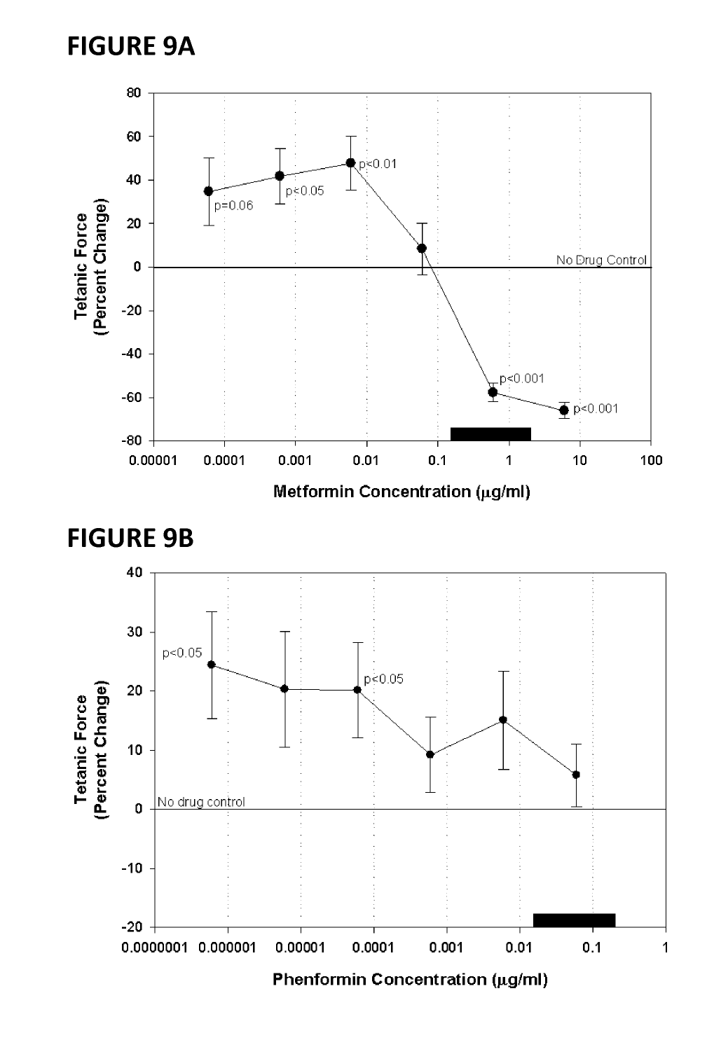

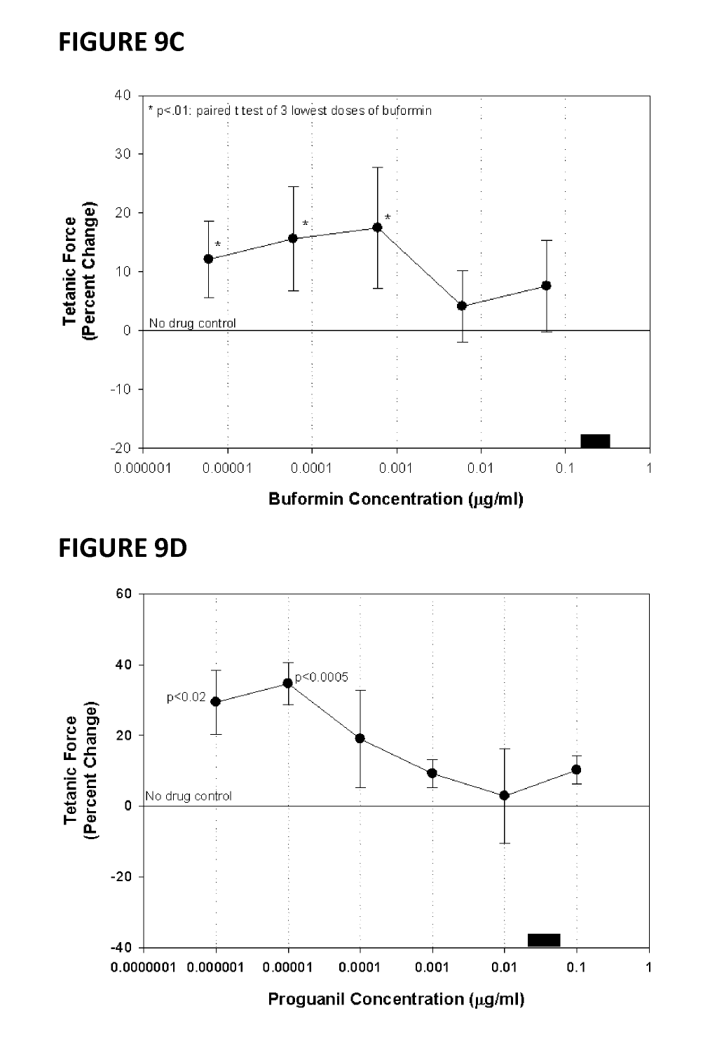

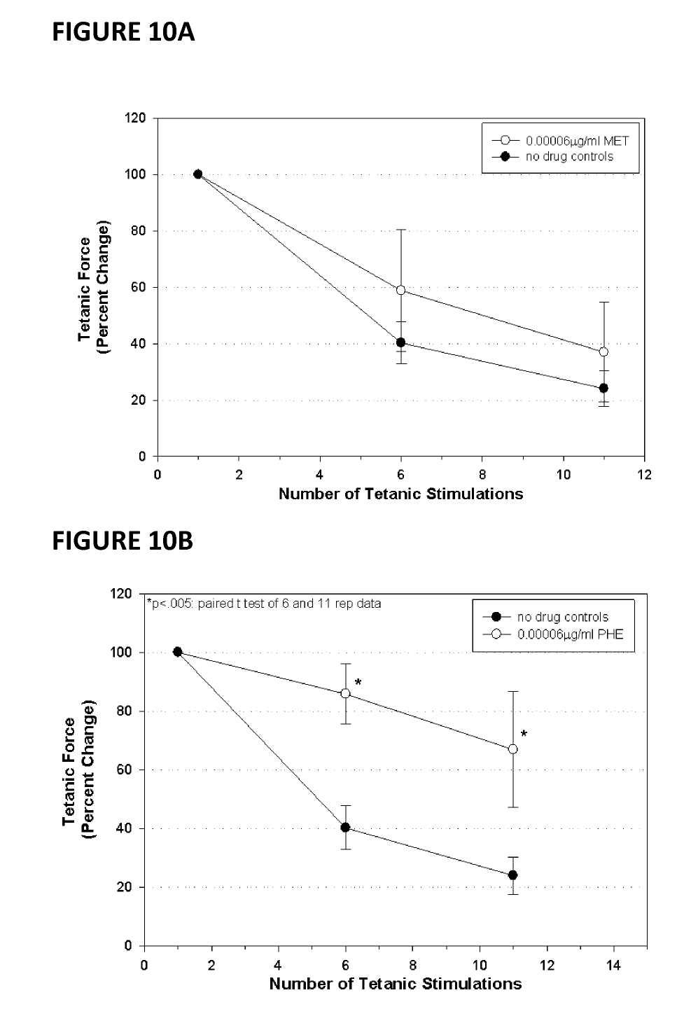

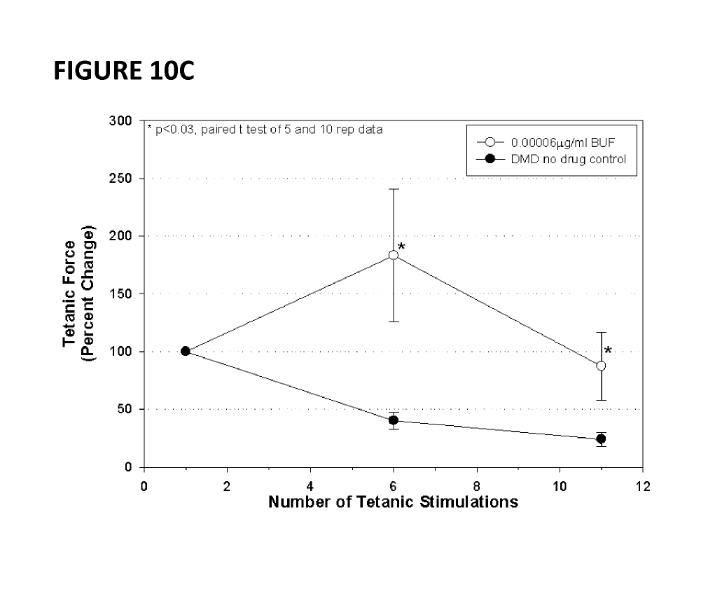

Metformin concentrations between 0.0006 μg/ml and 0.006 μg/ml significantly increased tetanic force generated by the muscle tissue (FIG. 9A). Similar results were obtained for buformin (FIG. 9C). Low concentrations of phenformin (<0.001 μg/ml) increased tetanic force (FIG. 9B). Proguanil concentrations of 0.00006 μg/ml and lower significantly increased tetanic forces generated by the muscle tissue (FIG. 9D). Low concentrations of metformin, phenformin, and buformin also decreased the rate of muscle fatigue with repetitive stimulations (FIGS. 10A-10C).

The results of these experiments indicate that biguanides increase skeletal muscle strength and reduce the rate of muscle fatigue in dystrophic human skeletal muscle tissue at concentrations at, or less than, mean plasma levels in subjects taking a biguanide for other disease indications.

### Other Embodiments

From the foregoing description, it is apparent that variations and modifications may be made to the invention described herein to adopt it to various usages and conditions. Such embodiments are also within the scope of the following claims.

All patents, patent applications, and publications mentioned in this specification are herein incorporated by reference to the same extent as if each independent patent, patent application, or publication was specifically and individually indicated to be incorporated by reference.

What is claimed is:

1. A method of treating muscular dysfunction, increasing muscle strength, or decreasing muscle fatigue in a subject in need thereof, said method comprising administering to said subject a therapeutically effective amount of a biguanide or a pharmaceutically acceptable salt thereof.

2. The method of claim 1, wherein said muscular dysfunction is associated with a condition or disease selected from the group consisting of muscular dystrophy, sarcopenia, cachexia, cancer, acquired immune deficiency syndrome, advanced organ failure, chronic obstructive pulmonary disease, rhabdomyolysis, disuse atrophy, incontinence, sepsis, neuromuscular disease, and congenital myopathy.

3. The method of claim 2, wherein said muscular dystrophy is Duchenne muscular dystrophy.

4-20. (canceled)

21. A method of treating or reducing the likelihood of developing cancer in a subject in need thereof or extending the lifespan of said subject, said method comprising administering to said subject a low dosage of a biguanide or a pharmaceutically acceptable salt thereof.

22. (canceled)

23. A pharmaceutical composition comprising a biguanide or a pharmaceutically acceptable salt thereof at a dosage unit of less than 250 milligrams.

24. (canceled)
